(19) United States
(12) Patent Application Publication
(10) Pub. No.: US 2026/0263371 A1
(43) Pub. Date: Sep. 10, 2026

(54) A LYOPHILIZED RNA COMPOSITION

(71) Applicant: eTheRNA immunotherapies NV, Niel (BE)

(72) Inventors: Iris Bombeke, Niel (BE); Katarina Bukara, Niel (BE); Phillip Challis, Niel (BE); George R. Dakwar, Niel (BE); Senne Dillen, Niel (BE); Diletta Esposito, Niel (BE); Eva Kickova, Niel (BE); Sarah Vandenberghe, Niel (BE); Saverio Vendemia, Niel (BE)

(73) Assignee: eTheRNA immunotherapies NV, Niel (BE)

(21) Appl. No.: 18/863,861

(22) PCT Filed: May 12, 2023
(86) PCT No.: PCT/EP2023/062722

§ 371 Date: Nov. 7, 2024

(30) Foreign Application Priority Data
May 12, 2022 (EP) 22172907.2

(51) Int. Cl. A61K 9/51; A61K 9/08; A61K 9/1271; A61K 9/19; A61K 31/7105; A61K 47/18; A61K 47/26

(52) U.S. Cl. CPC A61K 9/5123; A61K 9/08; A61K 9/1271; A61K 9/19; A61K 31/7105; A61K 47/18; A61K 47/26

(57) ABSTRACT

The invention is situated in the field of RNA formulation, in particular to lyophilization of RNA. More specifically, the invention relates to a composition that is lyophilized and a method to obtain a lyophilized composition. Moreover, the present invention provides the use of a reconstituted composition according to the invention in human and/or veterinary medicine.

### CROSS-REFERENCE TO RELATED APPLICATIONS

This application is a National Stage Application under 35 U.S.C. § 371 of International Application No. PCT/EP2023/062722, filed May 12, 2023, which International Application claims priority to European Application No. 22172907.2, filed May 12, 2022, each of which is incorporated herein by reference.

### FIELD OF THE INVENTION

The invention is situated in the field of RNA formulations, in particular to lyophilization of RNA. More specifically, the invention relates to a composition that is lyophilized and a method to obtain a lyophilized composition. Moreover, the present invention provides the use of a reconstituted composition according to the invention in human and/or veterinary medicine.

### BACKGROUND OF THE INVENTION

An mRNA-based vaccine is a new and promising platform that can be prepared, comparatively, in a short period of time. mRNA vaccines have the advantage that they do not deliver viral nucleic acids to the host nucleus—avoiding integration with the host DNA—and the injected mRNA also has a relatively short half-life in vivo. Despite these merits of efficacy and safety, instability and ultracold storage requirement of mRNA vaccines remain major limitations. Ultra-cold storage requirements and short shelf life slow down the distribution of vaccines mostly in areas of the world with limited resources.

The required storage conditions during manufacturing, shipping and at the end-user site are considered important characteristics of the mRNA vaccine drug product. Stability of mRNA formulations can be impacted, to some extent, by multiple factors such as excipients, pH, temperature as well as optimization of the mRNA sequence; appropriate vector selection; encapsulation of mRNA in lipid nanoparticles (LNP), and freeze-drying. Freeze-drying or lyophilization is a technique to improve stability of liquid vaccine formulations by first freezing the formulation and removing the aqueous solvent through a sublimation and desorption process [WO2012098358].

In a series of patents from CureVac, claims are made to maintenance of the activity of mRNA formulations (naked mRNA with and without protamine) upon freeze-drying and storage by using cryoprotectants such as sugars [WO2016/165831; WO2011/069528; U.S. Pat. No. 9,616,084]. It is claimed that all quality attributes analyzed during the experimental period (up to 36 months) meet the stability specifications of a stable and safe RNA medicament (i.e. appearance, RNA integrity, RNA content, pH value, and osmolality). In 2020, Zhao et al. published their findings on the performance of freeze-dried LNP-mRNA (encoding for luciferase) with different cryoprotectants compared to fresh LNPs. These authors demonstrated that the reconstituted material maintains the mRNA expression efficiency in mice as observed with in vivo bioluminescence imaging studies [Zhao et al. 2020].

In a recent study of BioNTech/Pfizer [Muramatsu et al. 2022], it was demonstrated that nucleoside-modified mRNA LNPs can be lyophilized, and the physiochemical properties of the lyophilized material do not significantly change for 12 weeks after storage at room temperature and for at least 24 weeks after storage at 4° C. Importantly, the authors observe no decrease in the mouse immunogenicity of a lyophilized influenza mRNA-LNP vaccine after storage at 4° C. for 24 weeks.

Even though some lyophilization protocols of mRNA-LNP formulations are described, there is still a need for improved lyophilisable compositions to achieve a stable product with long-term shelf-life and minor or no impact on biological activity or physicochemical properties of the mRNA vaccines such as cake appearance, presence of visible particles after reconstitution, mRNA content, encapsulation efficiency, particle size and polydispersity index (PDI), pH, and osmolality.

The present invention is intended to solve the above problems and an object of the present invention is to obtain a composition that is particularly suitable to lyophilize to provide a stable product and—in a lyophilized state—maintains its physiochemical properties and eventually of the mRNA-LNP vaccine. The object is solved by the subject matter of the claims. In particular, the object underlying the present invention is solved according to a composition comprising one or more RNA molecule(s), one or more lipid nanoparticle(s), one or more cryoprotectant(s), and Tris(hydroxymethyl)methylamine (TRIS); wherein the composition is lyophilized; and wherein the concentration of TRIS in the composition prior to lyophilization is 3 mM or less. In particular, the composition can be used in human and/or veterinary medicine.

The invention further provides a method of lyophilization comprising mixing RNA molecule(s), one or more lipid nanoparticle(s), a cryoprotectant and TRIS, thereby forming a composition according to the invention; and afterwards lyophilizing the composition by means of a freezing step, a primary drying step and a secondary drying step; wherein the primary drying step is performed at a temperature below the glass transition temperature of the maximally freeze-concentrated solution (Tg′).

The inventors have surprisingly found that the freeze-drying composition according to the invention is particularly suitable for the process of lyophilization. After completion of the lyophilization process, the lyophilized composition is characterized by (i) a high mRNA recovery after freeze drying and reconstitution, (ii) an excellent RNA integrity and (iii) a minimal increase in LNP particle size. Furthermore, the specific composition attributes to more favorable PDI values for the liquid formulation and ensures adequate buffering capacities. The process integrity and functionality of the formulation remains preserved after the freeze drying. In particular, storage stability is increased, in particular with respect to storage for extended periods and/or under non-cooling conditions. The shelf life in a frozen state reaches up to 36 months at −20° C., in a cold state (2-8° C.) up to 24 months at 4° C. and at room temperature up to 24 hours at 25° C. The method according to the invention can be used to produce a composition comprising RNA having the above-mentioned properties in a reproducible and cost-effective manner. The lyophilized composition comprising RNA according to the invention can advantageously be stored, shipped and applied, e.g. in the medical field (for example as a vaccine), without a cold chain, while the integrity and the biological activity of the RNA in the composition remain exceptionally high.

### SUMMARY OF THE INVENTION

In a first aspect, the present invention relates to a composition comprising: one or more RNA molecule(s), one or more lipid nanoparticle(s), one or more cryoprotectant(s), and Tris(hydroxymethyl)methylamine (TRIS); wherein said composition is lyophilized; and wherein the concentration of TRIS in the composition prior to lyophilization is 3 mM or less.

In a particular embodiment of the present invention, said cryoprotectant is selected from the list comprising: trehalose, maltose or sucrose, or a combination thereof; in particular sucrose.

In another aspect, the present invention relates to a composition comprising: one or more RNA molecule(s), one or more lipid nanoparticle(s), sucrose, and Tris(hydroxymethyl)methylamine (TRIS); wherein said composition is lyophilized; and wherein the concentration of TRIS in the composition prior to lyophilization is 3 mM or less.

In yet a particular embodiment, the concentration of the cryoprotectant in the composition prior to lyophilization is at least about 10% (w/v), in particular at least about 15% (w/v), more in particular at least about 20% (w/v), preferably at least about 15% (w/v).

In a specific embodiment, the concentration of sucrose in the composition prior to lyophilization is at least about 5% (w/v), in particular at least about 8% (w/v), more in particular at least about 10% (w/v).

In a particular embodiment, the concentration of sucrose prior to lyophilization is about and between 5% to about 20% (w/v), in particular about and between 5% to about 15% (w/v), more in particular about and between 8% to about 15% (w/v), even more in particular about and between 10% to about 15% (w/v).

In a further embodiment of the present invention, the concentration of TRIS in the composition prior to lyophilization is about and between 0.01 mM to about 3 mM, preferably about and between 0.1 mM to about 2 mM, more preferably about and between 0.5 mM and 1.5 mM, most preferably about 1 mM.

In a further embodiment of the present invention, the concentration of TRIS in the composition prior to lyophilization is about and between 0.01 mM to about 3 mM, preferably about and between 0.1 mM to about 3 mM, more preferably about and between 0.5 mM and 3 mM, most preferably about and between 1 mM to about 3 mM. Alternatively about and between 2 mM to about 3 mM

In a specific embodiment of the present invention, said lipid nanoparticle(s) comprise a PEG lipid, in particular a PEG2000 lipid, more in particular DMG-PEG2000.

In a specific embodiment, the lipid nanoparticle in the composition prior to lyophilization comprises about and between 40 mol % and 60 mol % of ionizable lipid, about and between 5 mol % and 15 mol % of phospholipid, about and between 20 mol % and 40 mol % of sterol, and at least 0.5 mol % of a PEG lipid.

In yet a further embodiment, the composition prior to lyophilization further comprises water or water for injection (WFI).

In a following embodiment of the present invention, said one or more RNA molecule(s) are linear or circular RNA molecule(s).

In yet a further embodiment, said one or more RNA molecule(s) are mRNA molecule(s).

In a further aspect, the present invention provides a composition for use in human and/or veterinary medicine.

In a preferred embodiment, the composition according to the invention is reconstituted before administration to a subject in need thereof.

In a specific embodiment, the reconstitution is performed using water in particular water for injection (WFI) or an aqueous solution of a salt, preferably TBS, more preferably TBS with 20 mM TRIS, most preferably TBS with 20 mM TRIS and 0.9% NaCl.

In yet a specific embodiment, the reconstituted composition has a pH of about 6 to about 8.

In yet a further aspect, the present invention provides a method of lyophilization of a composition comprising RNA molecules and lipid nanoparticles, said method comprising mixing RNA molecule(s), one or more lipid nanoparticle(s), a cryoprotectant and TRIS at a concentration of 3 mM or less in water or water for injection, thereby forming the composition according the invention; and lyophilizing the composition by means of a freezing step, a primary drying step and a secondary drying step; wherein said primary drying step is performed at a temperature below the glass transition temperature of the maximally freeze-concentrated solution (Tg′).

In a further embodiment of the method of the present invention, the primary during step is performed at a temperature of about and between −30° C. to about −50° C., preferably about and between −35° C. and about −45° C. in particular at about −40° C.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Physiochemical properties of lyophilized composition comprising 3 mM Tris and various amounts of sucrose prior to lyophilization. Results are presented for the Freeze-dried cake (FD cake) reconstituted in WFI compared to a −80° C. liquid control. (A) The RNA content (μg/ml). (B) Osmolality (mOsm/kg). (C) Encapsulation efficiency (%).

FIG. 2: Antibody response after Sars-Cov2 Omicron spike. Freeze-dried compositions were stored for 0 months (TO), 3 months (T3) or 5 months (T5) at different temperatures i.e. 2-8° C., 25° C. or 30° C. before reconstitution. Liquid controls were stored at −80° C. or at 2-8° C. To monitor the development of immune response in these mice, a submandibular blood collection was performed on D20 and D35. The concentration of anti-Omicron IgG in serum was determined by ELISA.

FIG. 3: Luciferase expression at 4 h, 24 h and 48 h after LNP injection. Injection was performed either with TBS or liquid −80° C. sample (controls) or freeze-dried compositions that were stored for 0 months (T0) (Panel A) or 5 months (T5) (Panel B) at different temperatures i.e. 2-8° C., 25° C. or 30° C. before reconstitution.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements, or method steps. The terms also encompass “consisting of” and “consisting essentially of”.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

In a first aspect, the present invention relates to a composition comprising: one or more RNA molecule(s), one or more lipid nanoparticle(s), one or more cryoprotectant(s), and Tris(hydroxymethyl)methylamine (TRIS); and wherein the concentration of TRIS in the composition prior to lyophilization is 3 mM or less.

As used herein, the term ‘composition’, may refer to any mixture of two or more products or compounds for the purpose of long-term stabilization such as prevention of denaturation or aggregation over the expected shelf life. In the context of the present invention, the term ‘lyophilized composition’ of the present invention refers to the end-product after the lyophilization process in the form of a lyophilized powder or solid form. On the other hand, the term ‘reconstituted composition’ is used for the lyophilized composition in an aqueous form after reconstitution in for example water and ready to be administered to a subject in need thereof. Lastly, the present invention also refers to a ‘freeze drying composition’ which is a composition prior to the lyophilization process which may be in the form of a solution, a suspension, liquid, or aqueous formulation. Concentrations, amounts, ratios, proportions etc. of for example TRIS, cryoprotectant(s), RNA molecule(s), LNP(s) as used herein relate to the composition after lyophilization (i.e. lyophilized composition) unless otherwise stated. More specifically, in those cases the application will refer to concentrations, amounts, ratios, proportions etc. of the composition prior to lyophilization (i.e. freeze drying composition).

In the context of the present invention, the term ‘lyophilisation’ (also known as freeze drying) is applied to maintain or improve a product's shelf life, making it more convenient to be stored, distributed, and transported. A freeze-drying process typically comprises three consecutive steps: i) a freezing step wherein a solvent (such as for example water) crystallizes to ice, ii) a primary drying step wherein the solvent is removed under vacuum by sublimation, iii) a secondary drying step wherein most of the unfrozen solvent is removed by diffusion and desorption.

In the context of the present invention, the term ‘TRIS’ or tris(hydroxymethyl)aminomethane, or known during medical use as tromethamine or THAM, is to be understood as an organic compound with the formula (HOCH₂)₃CNH₂. It is routinely uses as a component of buffer solutions such as in TAE and TBE buffers, especially for solutions of nucleic acids. Tris is frequently used to increase permeability of cell membranes or as a compound to metal ions in a solution. As used herein, TRIS acts as a buffering agent maintaining the solution pH in an acceptable range prior to lyophilization. Many other buffering agents covering a wide pH range are available for selection in formulations for example, acetate, citrate, glycine, histidine, phosphate (sodium or potassium), and diethanolamine and therefore might be suitable as well in the context of the present invention. Although any pH adjuster may be used, the use of tris(hydroxymethyl)aminomethane hydrochloride is preferred.

In some embodiments, said concentration of TRIS in the composition prior to lyophilization may be about 3 mM or less such as about 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 mM.

In a particular embodiment, the concentration of TRIS in the composition prior to lyophilization is preferably about 0.5 mM, about 1 mM or about 1.5 mM.

In a further embodiment of the present invention, the concentration of TRIS in the composition prior to lyophilization is about and between 0.01 to about 3 mM, preferably about and between 0.1 mM to about 2 mM, more preferably about and between 0.5 mM and 1.5 mM, most preferably about 1 mM.

In another embodiment, the concentration of TRIS in the composition prior to lyophilization is about and between 0.01 to about 3 mM, preferably about and between 0.1 mM to about 3 mM, more preferably about and between 0.5 mM and 2 mM, most preferably about and between 1 mM to about 3 mM. Alternatively about and between 2 mM to about 3 mM.

In a specific embodiment, the concentration of TRIS in the composition prior to lyophilization is about 1 mM, about 2 mM or about 3 mM.

According to the present invention, the freeze-drying composition also comprises at least one cryoprotectant. As used herein and unless otherwise specified, the term “cryoprotectant” refers to an excipient and is to be understood as a penetrating or non-penetrating substance used to protect a composition and its associated compounds from freezing damage due to for example ice formation. Conventional cryoprotectants are commonly glycols (alcohols containing at least two hydroxyl groups) and sugars, such as ethylene glycol, propylene glycol, glycerol and trehalose. Generally, a sugar that is preferred in this context, has a high water displacement activity and a high glass transition temperature. In particular, sucrose and trehalose are non-reducing sugars that preserves the structural integrity of the cells during freezing and thawing in a non-toxic manner.

In a specific embodiment, the cryoprotectant may be selected from the list comprising sodium citrate, sodium chloride, sorbitol, polysorbate, trehalose, mannose, mannitol, maltose, sucrose, glucose, fructose, lactose, histidine, arginine, lysine, dextran, maltodextrin, cyclodextrins, polyvinylpyrrolidone (PVP), glycine, glycerol, polyethylene glycol (PEG), propylene glycol, and/or mixtures thereof.

In a particular embodiment of the present invention, said cryoprotectant is selected from the list comprising: trehalose, maltose or sucrose, or a combination thereof; in particular sucrose.

In a specific embodiment, the cryoprotectant is sucrose.

Hence, the present invention relates to a composition comprising: one or more RNA molecule(s), one or more lipid nanoparticle(s), sucrose, and Tris(hydroxymethyl)methylamine (TRIS); and wherein the concentration of TRIS in the composition prior to lyophilization is 3 mM or less.

In a particular embodiment, the composition of the present invention prior to lyophilization comprises at least about 0.01% (w/v), at least about 0.1% (w/v), at least about 0.5% (w/v), at least about 1% (w/v), at least about 2.5% (w/v), at least about 5% (w/v), at least about 10% (w/v), at least about 15% (w/v), at least about 20% (w/v), at least about 25% (w/v), at least about 30% (w/v), in particular at least about 15% (w/v) of a cryoprotectant.

In yet a particular embodiment, the concentration of the cryoprotectant in the composition prior to lyophilization is about and between 0.01% to about 30% (w/v), preferably about and between 10% to about 20% (w/v), most preferably about 15% (w/v).

In another embodiment, the concentration of sucrose prior to lyophilization is about and between 5% to about 20% (w/v) sucrose such as about 5%, 6%, 8%, 9%, 10% to about 15%, 16%, 17%, 18%, 19%, 20% (w/v) sucrose.

In another embodiment, the concentration of sucrose prior to lyophilization is about and between 5% to about 20% (w/v), in particular about and between 5% to about 15% (w/v), more in particular about and between 8% to about 15% (w/v), even more in particular about and between 10% to about 15% (w/v). In one embodiment, the concentration of the cryoprotectant in the composition prior to lyophilization is about 5%, 6%, 7%, 8%, 9%, 10%, 11%, 12%, 13%, 14%, 15%, 16%, 17%, 18%, 19%, 20%, 21%, 22%, 23%, 24%, 25% (w/v).

In one embodiment, the concentration of the cryoprotectant in the composition prior to lyophilization is 15% (w/v)

In another embodiment, the concentration of sucrose in the composition prior to lyophilization is about 5%, 6%, 7%, 8%, 9%, 10%, 11%, 12%, 13%, 14%, 15%, 16%, 17%, 18%, 19%, 20%, 21%, 22%, 23%, 24%, 25% (w/v).

In a specific embodiment, the concentration of sucrose in the composition prior to lyophilization is 15% (w/v).

In a further embodiment, the composition prior to lyophilization of the present invention comprises a concentration of 1 mM of TRIS and 15% sucrose (w/v).

In yet a further embodiment, the composition prior to lyophilization of the present invention comprises a concentration of 3 mM or less of TRIS and about and between 5% to about 20% (w/v) sucrose such as about 5%, 6%, 8%, 9%, 10% to about 15%, 16%, 17%, 18%, 19%, 20% (w/v).

In another embodiment, the composition prior to lyophilization of the present invention comprises a concentration of 3 mM or less of TRIS and about and between 5% to about 20% (w/v) sucrose such as about and between 8% to about 20% (w/v), about and between 8% to about 15% (w/v), about and between 10% to about 15% (w/v) sucrose.

In the context of the present invention, the composition according to the invention comprises at least one or more RNA molecule(s) that is delivered in one or more vehicles comprising lipids, liposomes, lipid nanoparticles, polymers or polymer-based nanoparticles.

As used herein, lipids are referred to as any component from the category fatty acids, glycerolipids, glycerophopholipids, sphingolipids, sterols, prenols, saccharolipids, polyketides.

Further, in the context of the present invention, liposomes are spherical vesicle structures having at least one lipid bilayer that forms in the shape of a hollow sphere encompassing an aqueous phase. As such, any cargo of interest such as nucleic acid containing formulations, pharmaceutical drugs, proteins/peptides can be encapsulated within liposomes in either the aqueous compartment (if it is water-soluble/hydrophilic) or within the lipid bilayer (if fat-soluble/lipophilic).

In the context of the present invention, by means of the term “lipid nanoparticle”, or LNP, reference is made to a nanosized particle composed of one or more lipids, e.g. a combination of different lipids particularly useful in encapsulating a broad variety of nucleic acids (RNA and DNA) as a drug or vaccine, in a non-aqueous core. More specifically, the lipid nanoparticle is generally spherical in shape and consists of a solid lipid core stabilized by a surfactant. The core lipids can be fatty acids, acylglycerols, waxes, and mixtures thereof. Biological membrane lipids such as phospholipids, sphingomyelins, bile salts (sodium taurocholate), and sterols (cholesterol) can be utilized as stabilizers. Possible lipids used in the LNP can be for example, but not limited to at least one phospholipid, at least one modified lipid, such as a PEG lipid, (e.g PEG2000 lipid), at least one ionisable lipid, and at least one sterol. The lipid nanoparticles of the disclosure and the compositions thereof are generally known in the art.

In the context of the present invention, the term “PEG lipid” or alternatively “PEGylated lipid” is meant to be any suitable lipid modified with a PEG (polyethylene glycol) group. Particularly suitable PEG lipids in the context of the present invention are characterized in being diC14-PEG lipids. Where in the context of the invention, the term C14-PEG lipids is used, this is meant to be diC14-PEG lipids, i.e. lipids having 2 C14 lipid tails. C14-PEG lipids contain a polyethylene glycol moiety, which defines the molecular weight of the lipids, as well as a fatty acid tail comprising 14 C-atoms. For example, PEG lipids in the context of the present invention can be diC14-PEG lipids, such as for example DMG-PEG, more in particular DMG-PEG2000 (1,2-dimyristoyl-rac-glycero-3-methoxypolyethylene glycol-2000), or DMPE-PEG, more in particular DMPE-PEG2000 (1,2-dimyristoyl-sn-glycero-3-phosphoethanolamine-N-[methoxy(polyethylene glycol)-2000). Alternatively, also longer chain PEG lipids, such as diC16-lipids or diC18-lipids can be suitably used. In a particular embodiment, said diC18-PEG2000 lipid is selected from the list comprising: a (distearoyl-based)-PEG2000 lipid such as DSG-PEG2000 lipid (2-distearoyl-rac-glycero-3-methoxypolyethylene glycol-2000) or DSPE-PEG2000 lipid (1,2-distearoyl-sn-glycero-3-phosphoethanolamine-N-[methoxy(polyethylene glycol)- 2000]); or a (dioleolyl-based)-PEG2000 lipid such as DOG-PEG2000 lipid (1,2-Dioleolyl-rac-glycerol) or DOPE-PEG2000 lipid (1,2-dioleoyl-sn-glycero-3-phosphoethanolamine-N-[amino(polyethylene glycol)-2000]) or PEG5000 lipids.

In a specific embodiment, said PEG2000 lipid comprises at least 3 oxygen atoms in the fatty acid tail of said lipid. Examples of such PEG lipids are DMG-PEG2000, DSPE-PEG2000.

In a specific embodiment of the present invention, said PEG2000 lipid is DMG-PEG2000.

In the context of the present invention the term “ionisable” (or alternatively cationic) in the context of a compound or lipid means the presence of any uncharged group in said compound or lipid which is capable of dissociating by yielding an ion (usually an H+ ion) and thus itself becoming positively charged. Alternatively, any uncharged group in said compound or lipid may yield an electron and thus becoming negatively charged. As used herein, any type of ionizable lipid can suitably be used. For example, suitable ionizable lipids are ionizable amino lipids which comprise 2 identical or different tails linked via an S—S bond.

In the context of the present invention, the term “phospholipid” is meant to be a lipid molecule consisting of two hydrophobic fatty acid “tails” and a hydrophilic “head” consisting of a phosphate group. The two components are most often joined together by a glycerol molecule, hence, the phospholipid of the present invention is preferably a glycerol-phospholipid. Furthermore, the phosphate group is often modified with simple organic molecules such as choline (i.e. rendering a phosphocholine) or ethanolamine (i.e. rendering a phosphoethanolamine).

In the context of the present invention, the term “sterol”, also known as steroid alcohol, is a subgroup of steroids that occur naturally in plants, animals and fungi, or can be produced by some bacteria. In the context of the present invention, any suitable sterol may be used, such as selected from the list comprising cholesterol, ergosterol, campesterol, oxysterol, antrosterol, desmosterol, nicasterol, sitosterol and stigmasterol; preferably cholesterol.

In a particular embodiment, said LNP comprises about and between 10 mol % and 60 mol % of said ionisable lipid; preferably about and between 40 mol % and 60 mol %.

In yet another specific embodiment, said LNP comprises about and between 15 mol % and 50 mol % of sterol; preferably about and between 20 mol % and 40 mol %.

In a further embodiment, said LNP comprises about and between 0.5 mol % and 10 mol % of said PEG2000 lipid; preferably about and between 0.5 mol % and 5 mol %.

In another embodiment, said LNP comprises at least 0.5 mol %, such as at least 1 mol %, such as at least 1.5 mol %, such as at least 2 mol %, such as at least 2.5 mol %, such as at least 3 mol % of said PEG2000 lipid.

In another specific embodiment, said LNP comprises about and between 5 mol % and 40 mol % of said phospholipid; preferably about and between 5 mol % and 15 mol %.

In a specific embodiment, the lipid nanoparticle in the composition prior to lyophilization comprises about and between 40 mol % and 60 mol % of ionizable lipid, about and between 5 mol % and 15 mol % of phospholipid, about and between 20 mol % and 40 mol % of sterol, and at least 0.5 mol % of said PEG2000 lipid.

In yet a further embodiment, the composition prior to lyophilization further comprises water or water for injection (WFI).

In the context of the present invention, the term ‘water for injection’ is to be understood as a sterile, nonpyrogenic, solute-free preparation of distilled water purified by distillation or reverse osmosis with a pH of about 5.0 to 7.0. It is for use only as a sterile solvent or diluent vehicle for drugs or solutions suitable for parenteral administration, and intended to be used in the production of drug products or for injection.

In a following embodiment of the present invention, said one or more RNA molecule(s) are linear or circular RNA molecule(s).

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. In particular, the term refers to single stranded RNA, but may also refer to double stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

In yet a further embodiment, said one or more RNA molecule(s) are mRNA molecule(s).

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro. Preferably, mRNA is produced by in vitro transcription using a DNA template. In one embodiment of the invention, the RNA is obtained by in vitro transcription or chemical synthesis. The in vitro transcription methodology is known to the skilled person. For example, there is a variety of in vitro transcription kits commercially available.

To avoid any misunderstanding, the composition according to the present invention comprises LNPs further comprising one or more mRNA molecule(s), or they may comprise a multitude of mRNA molecules, such as a combination of one or more mRNA molecules encoding immune modulating proteins and/or one or more mRNA molecules encoding antigen- and/or disease-specific proteins.

In a very specific embodiment, the composition comprises one or more mRNA molecules encoding at least one immunostimulatory protein selected from the list comprising CD40L, CD70 and caTLR4.

Accordingly, the invention provides a lyophilized composition comprising mRNA molecules encoding CD40L, mRNA molecules encoding CD70, mRNA molecules encoding caTLR4, wherein said mRNA molecules are formulated in lipid nanoparticles, and wherein said composition further comprises one or more cryoprotectant(s), and TRIS in a concentration of 3 mM or less prior to lyophilization. In a further aspect, the present invention provides a composition for use in human and/or veterinary medicine.

In a preferred embodiment, the lyophilized composition according to the invention is reconstituted before administration to a subject in need thereof. This reconstituted form of the composition is further termed ‘reconstituted composition’. In a particular embodiment, the reconstituted composition according to the invention is a pharmaceutical composition comprising the lyophilized composition and at least one or more pharmaceutically acceptable agents such as excipients, carriers, diluents.

In the context of the present invention, by means of the term “pharmaceutical composition” reference is made to a composition having pharmaceutical properties such as a vaccine (or vaccine composition). In other words, reference is made to a composition providing for a pharmacological and/or physiological effect. In some embodiments, the pharmaceutically acceptable agents include, but are not limited to, biocompatible vehicles, adjuvants, additives, and diluents to achieve a composition usable as a dosage form. Additional suitable pharmaceutical carriers and diluents, as well as pharmaceutical necessities for their use, are described in Remington's Pharmaceutical Sciences. After freeze-drying, the resulting dried composition may be reconstituted using any suitable medium/buffer, such as but not limited to water, water for injection, Tris buffered saline (TBS) and/or Phosphate buffered saline (PBS).

In a specific embodiment, the reconstitution is performed using water or an aqueous solution of a salt, preferably TBS, more preferably TBS with 20 mM TRIS, most preferably TBS with 20 mM TRIS and 0.9% NaCl.

In a specific embodiment, the reconstitution is performed using water for injection (WFI). In yet a specific embodiment, the reconstituted composition has a pH of about 6 to about 8.

In a particular embodiment, the reconstituted composition has a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, preferably a pH of about 7.0, preferably about 7.4.

The invention provides a method for the prophylaxis and treatment of human and veterinary disorders, by administering a composition or a pharmaceutical composition to a subject in need thereof.

In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom.

In a particular embodiment, the present invention provides a pharmaceutical composition as defined herein, which after reconstitution is suitable for use in parenteral administration; more in particular for use in intravenous, intratumoral, intradermal, intraperitoneal, intramuscular or intranodal administration, preferably intramuscular administration.

In a specific embodiment, the present invention provides a reconstituted composition or a pharmaceutical composition for use in the treatment or prevention a pathogen in a subject; in particular a virus pathogen, more in particular the SARS-CoV-2 virus.

In another embodiment, a reconstituted composition or pharmaceutical composition as defined herein is provided for use in the prevention and/or treatment of cell proliferative disorders.

In yet a further aspect, the present invention provides a method of lyophilization comprising: a) mixing RNA molecule(s), one or more lipid nanoparticle(s), a cryoprotectant and TRIS, thereby forming the composition prior to lyophilization according the invention; and b) lyophilizing said composition by means of a freezing step, a primary drying step and a secondary drying step; wherein said primary drying step is performed at a temperature below the glass transition temperature of the maximally freeze-concentrated solution (Tg′).

As used herein, the term ‘glass transition temperature’ (Tg) is to be understood as the temperature at which the frozen material changes from a glassy (brittle) to rubbery (flexible of soft) state. Typically, in spray-freeze-drying methods of amorphous materials, two different types of glass transition temperatures have been considered: (i) glass transition temperature of the maximally freeze-concentrated solution (Tg′), which is relevant in the frozen solution state; (ii) glass transition temperature of the drying solid phase (Tg) that holds good after the primary drying begins. In particular, in order to decide on the shelf temperature during the primary drying as to avoid collapse, it is more relevant to consider Tg′ than Tg. The shelf life of lyophilized proteins can be affected by the product temperature during the primary drying step. Performing primary drying below the Tg′ will make it possible to maintain the physical stability of the lyophilized product (i.e., the shape of the cake).

In a particular embodiment, said primary drying can also be performed at a temperature below the collapse temperature (Tc).

As used herein, the term ‘collapse temperature’ (Tc) is to be understood as the temperature at which a material softens to the point at which it would not be able to support its structure. The collapse phenomenon has a detrimental effect on the properties of the final freeze-dried product, leading to volatile loss during storage, poor reconstitution behaviour, non-uniform moisture distribution, and extensive caking. Typically, the Tc of a material tends to be different to the temperature applied during drying. In some embodiments, the product temperature during primary drying is maintained 2-5° C. below its Tc to avoid collapse and maintain an elegant cake structure. For example, a freeze-dry process wherein the product temperature is −25° C. and the Tc is −20° C. results in good appearance. Usually, the values of the collapse temperature are higher than the Tg′ values by 1-3° C. Generally speaking, the target product temperature during the primary drying stage of an optimized lyophilization process is several degrees below a critical threshold value corresponding to the glass transition temperature of the freeze-concentrated phase (Tg′). The temperatures defined herein with respect to the method according to the invention typically refer to the respective temperatures in the freeze-drying chamber. Depending on the type of instrument, the temperature in the freeze-drying chamber may be determined by different means.

In a particular embodiment, said primary drying is performed at a temperature that is about 0.5° C., about 1° C., about 2° C., about 3° C., about 4° C., about 5° C., about 6° C., about 7° C., about 8° C., about 9° C., about 10° C. below the collapse temperature (Tc). As an example, for a product described in the present application, the collapse temperature of the composition is −33° C., the Tg′ is approximately −35° C. and the primary drying temperature is performed at −40° C.

In a specific embodiment of the method of the present invention, the primary drying step is performed at about and between −30° C. to about −50° C., preferably about and between −35° C. and about −45° C., in particular at about −40° C.

In preferred embodiments of the invention, the primary drying temperature is equal to or at least 0.5° C., 1° C., 2° C., 3° C., 4° C., 5° C., 6° C., 7° C., 8° C., 9° C., 10° C., 11° C., 12° C., 13° C., 14° C. or 15° C. lower than the Tg′ of a given composition.

Preferably, the primary drying temperature in step b) of the method according to the invention is below −25° C., more preferably below −30° C. and most preferably below −35° C. Further preferably, the primary drying temperature in step b) of the method according to the invention is in a range from −55° C. to −25° C., preferably from −50° C. to −30° C., more preferably in a range from −45° C. to −35° C. and most preferably in a range from −43° C. to −37° C. In a particularly preferred embodiment, the primary drying temperature is about −40° C.

In a particular embodiment, the primary drying step temperature ramps up to the secondary drying temperature. In some embodiments, the primary drying temperature starts about and between −30° C. to about −50° C., preferably about and between −35° C. and about −45° C., in particular at about −40° C. and ramps up to about 10° C. wherein afterwards the secondary drying step starts.

The Tg is typically determined empirically. Methods for determining the glass transition temperature of a substance or composition are known in the art and comprise, for example, by using a freeze-drying microscope, a differential thermal analyser (e.g. differential scanning calorimetry) or an electric impedance analyser (dielectric resistance analysis). The freezing temperature is preferably pre-determined by selecting a temperature equal to or below the Tg of a given composition.

In preferred embodiments of the invention, the freezing temperature is equal to or at least 0.5° C., 1° C., 2° C., 3° C., 4° C., 5° C., 6° C., 7° C., 8° C., 9° C., 10° C., 11° C., 12° C., 13° C., 14° C. or 15° C. lower than the Tg of a given composition. For example, if the primary drying step is performed at −40° C., which is below the Tg of a given composition, the freeze-drying step can be performed at for example −50° C.

Preferably, the freezing temperature in step b) of the method according to the invention is below −30° C., more preferably below −35° C. and most preferably below −40° C. Further preferably, the freezing temperature in step b) of the method according to the invention is in a range from −65° C. to −35° C., preferably from −60° C. to −40° C., more preferably in a range from −55° C. to −45° C. and most preferably in a range from −53° C. to −47° C. In a particularly preferred embodiment, the freezing temperature is about −50° C.

### EXAMPLES

The invention will now be illustrated by means of the following examples. It will be understood by those skilled in the art that various changes and modifications can be easily made without departing from the technical spirit or essential features of the present invention. Therefore, it is to be understood that the below-described examples are illustrative in all aspects and do not limit the scope of the invention in any way.

### Materials and Methods

### Raw Materials:

DSPC, cholesterol and DMG-PEG2000 were purchased from Avanti Polar Lipids Inc. (Birmingham, Alabama, USA). The proprietary ionizable was synthesized at eTheRNA Immunotherapies or by an external party. eGFP mRNA was produced at eTheRNA Immunotherapies, Niel, Belgium.

### Preparation of the Freeze-Drying Composition

Lipid nanoparticles (LNPs) encapsulating EGFP mRNA were produced using a mixing device where a solution containing EGFP mRNA in sodium acetate buffer (100 mM, pH4) is mixed with lipid solution containing a ionizable lipid (50% mol %), helper lipid (10% mol %), cholesterol (38.5% mol %) and DMG-PEG2000 at a pre-defined flow rate ratio. The post-mixing bulk was diluted in water for injection (WFI) and processed via tangential flow filtration (TFF) in three sub-steps: concentration, diafiltration, and a final concentration. The obtained bulk from the TFF process is diluted with the cryoprotectant to obtain a final Tris buffer concentration of 1 mM in 15% sucrose. The solution is then sterile filtered using a 0.22 μm Polyether sulfone (PES) filter. T.

### Lyophilization Process

Vials containing 800 μL of the above-mentioned lipid composition encapsulating eGFP mRNA in 1 mM Tris and 15% sucrose are subjected to a freeze-drying cycle using as described below:
1. Temperature calibration at 20° C.
2. Freezing to −50° C. for 100 minutes at a rate of (0.7° C./min), to achieve a temperature which is below Tg′
3. Temperature of −50° C. is maintained for 60 minutes
4. Primary drying sub-step 1: the temperature is maintained at −50° C. for 15 minutes and the vacuum is switched on the pressure set value: 0.0720 mbar
5. Primary drying sub-step 2: the temperature is maintained at −50° C. for 3 hours. Vacuum set value: 0.0720 mbar
6. Primary drying sub-step 3: the temperature is increased to −40° within 2 hours at a rate of 0.08° C./min, while maintaining the vacuum set value constant: 0.0720 mbar
7. Primary drying sub-step 4: Temperature is maintained at −40° C. for 70 hours, and the same vacuum set value −0.0720 mbar
8. Primary drying sub-step 5: the temperature is increased to 10° C. within 10 hours at a rate of 0.08° C./min. Vacuum set value maintained at 0.0720 mbar
9. Secondary drying sub-step 1: the temperature is maintained at 10° C. for 15 minutes, while the vacuum set value is lowered to 0.0100 mbar
10. Secondary drying sub-step 2: the temperature is maintained at 10° C. for 5 hours and 50 minutes using a vacuum set value of 0.0100 mbar.

### Results

### Example 1—Freeze-Drying Composition Selection—Phase 1

Freeze-drying composition screening was conducted to select the optimal cryoprotectant formulation that results in the best cake aspects and quality attributes following reconstitution. TBS and WFI were tested in combination with trehalose and/or sucrose. Of note, samples freeze-dried in WFI were reconstituted with TBS, while samples freeze-dried in TBS were reconstituted in WFI. The data in Table 1 suggest that sucrose outperforms trehalose as a cryoprotectant, while no improvement in cake aspect and particle quality attributes were observed using 20% or 25% of cryoprotectant. Therefore, further formulation optimization was performed using 15% sucrose as demonstrated in example 2.

TABLE 1
Summary table on selection criteria for freeze drying composition selection.

| | | | Particle | | | |
|---|---|---|---|---|---|---|
| | | Visual aspect | size | | Recovery | mRNA |
| | Cake | after | (Z-average | | mRNA | Encapsulation |
| Freeze drying composition | aspect | reconstitution | diameter, nm) | PDI | (%) | efficiency (%) |
| 15% sucrose TBS, pH 7.4 | A | X | 165.6 | 0.159 | 87% | 94% |
| 20% sucrose TBS, pH 7.4 | A | X | 137.0 | 0.146 | 68% | 90% |
| 15% trehalose TBS, pH 7.4 | A | Y | 355.5 | 0.503 | 60% | 64% |
| 20% trehalose TBS, pH 7.4 | | Y | 371.3 | 0.434 | 65% | 66% |
| 15% sucrose WFI | A | Z | 97.98 | 0.242 | 48% | 83% |
| 20% sucrose WFI | B | Z | 114.1 | 0.230 | 72% | 83% |
| 25% sucrose WFI | B | Z | 107.6 | 0.201 | 68% | 85% |
| 15% trehalose WFI | A | Z | 135.4 | 0.236 | 75% | 83% |
| 20% trehalose WFI | B | Z | 146.9 | 0.241 | 69% | 83% |

A: Homogenous, coherent cake, no collapse or melting.
B: Bad cake, melting present.
X: Translucent, homogeneous solution without visible particles.
Y: Translucent, foamy solution without visible particles.
Z: Translucent, foamy solution, presence of visible particles.

### Example 2—Freeze-Drying Composition Selection—Phase 2

The freeze-drying composition comprising 15% sucrose was further optimized to improve mRNA encapsulation efficiency and recovery, decrease the PDI value and obtain better control of particle size following reconstitution.

Several formulations without or containing increased TRIS concentrations were tested in the presence of 15% sucrose and subjected to freeze-drying as detailed above under lyophilization process, and reconstituted with TBS. Among the four tested conditions, 1 mM Tris with 15% sucrose resulted in the best particle quality attributes: particle size is retained and mRNA encapsulation efficiency remains high.

TABLE 2
Summary table on selection criteria for formulation selection.

| | | Visual | Particle size | | Recovery | |
|---|---|---|---|---|---|---|
| | Cake | aspect after | (Z-average | | mRNA | Encapsulation |
| Freeze-drying composition | aspect | reconstitution | diameter, nm) | PDI | (%) | efficiency (%) |
| 15% sucrose WFI | A | X | 85.87 | 0.102 | 93% | 90% |
| 15% sucrose 1 mM TRIS | A | X | 90.28 | 0.119 | 86% | 91% |
| 15% sucrose 5 mM TRIS | A | X | 111.0 | 0.123 | 80% | 86% |
| 15% sucrose 20 mM TRIS | A | X | 111.2 | 0.162 | 86% | 84% |

A: Homogenous, coherent cake, no collapse or melting.
X: Translucent, homogeneous solution without visible particles.

### Example 3—Freeze-Drying Composition Selection—Phase 3

To assess whether concentrations of Tris below 5 mM such as 3 mM were also suitable as freeze-drying composition, a further optimization experiment was performed. Different amount of sucrose (5%, 8%, 10%, 15% and 20% w/v) were tested in a composition comprising 3 mM Tris before lyophilization (Table 3). Results are presented for the Freeze-dried cake (FD cake) reconstituted in WFI compared to a −80° C. liquid control

TABLE 3
Summary table on selection criteria for
freeze drying composition selection.

| | | Particle size | | mRNA |
|---|---|---|---|---|
| Freeze drying | Cake | (Z-average | | Encapsulation |
| composition | aspect | diameter, nm) | PDI | efficiency (%) |
| 3 mM Tris, | AB | 123.2 | 0.198 | 88.4% |
| 5% sucrose, | | | | |
| 3 mM Tris, | AB | 111.5 | 0.18 | 90.5% |
| 8% sucrose | | | | |
| 3 mM Tris, | A | 110.3 | 0.173 | 90.4% |
| 10% sucrose | | | | |
| 3 mM Tris, | A | 107 | 0.157 | 91.1% |
| 15% sucrose | | | | |
| 3 mM Tris, | C | 113.9 | 0.157 | 88.4% |
| 20% sucrose | | | | |

A: Homogenous, coherent cake, no collapse or melting.
B: Some shrinkage at the bottom.
C: Bad cake, melting present.

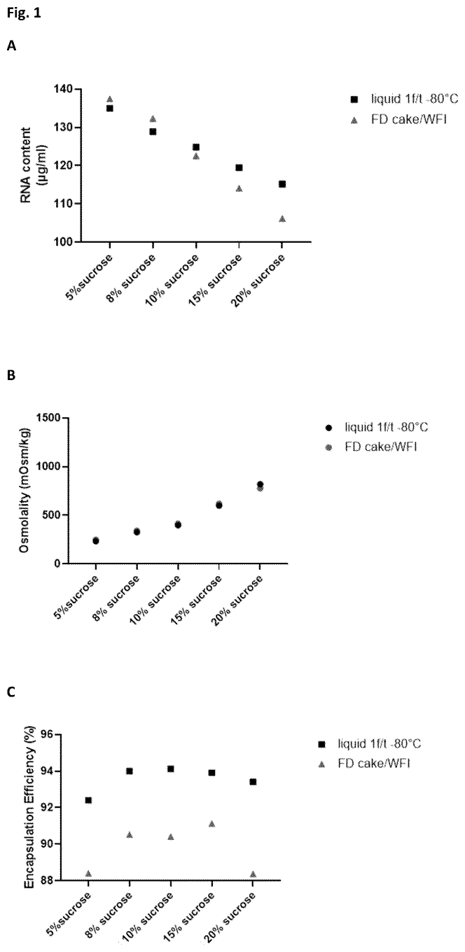

The size of various LNPs remained within the range between 107-123 nm. PDI remained very low, meaning below value of 0.2. mRNA content decreased (FIG. 1A) and osmolality increased (FIG. 1B) by increasing amount of sucrose while pH remained consistent at pH 7.4) (data not shown).

Compared with the −80° C. liquid control, the encapsulation efficiency was lower but showed consistency between formulations with 8-15% w/v sucrose (FIG. 1C). Moreover, these formulations showed similar encapsulation efficiencies as a formulation with 1 mM Tris, 15% sucrose (Table 2). As clearly derivable from the Table 2 and 3, formulations comprising either 1 mM Tris or 3 mM Tris with varying amounts of sucrose perform equally well and are also superior to a formulation comprising 20 mM Tris or 5 mM Tris and 15% sucrose.

### Example 4—Stability Study of 1 mM Tris Composition

To investigate the stability of the lyophilized composition, the selected freeze-drying composition (1 mM Tris, 15% Sucrose) was prepared, freeze-dried and placed at two different storage conditions: (1) 2-8° C. and (2) 25° C. for 3 months. A control sample of liquid (non-lyophilized) material containing the drug product was used in this study. At each time point, different quality attributes of the particles were tested after reconstitution with TBS (20 mM Tris, 0.9% NaCl). As highlighted in Table 4 and Table 5, all tested quality attributes remain stable as a function of time for the cakes stored at 2-8° C. Among these are the critical quality attributes of particle size, encapsulation efficiency, and mRNA content. For the cakes stored at 25° C. (accelerated storage condition), the quality attributes remain stable over time, except for a slight drop in the mRNA content. Worth mentioning also, that mRNA integrity and in-vitro functionality of the reconstituted cakes remains is retained over time compared to T0. This suggests that the mRNA remains intact and is not destabilized during the freeze-drying process as well as upon in the lyophilized form upon storage at 2-8° C. Furthermore, it could be deduced the lipid nanoparticle was not destabilized.

To conclude, the freeze-drying composition composed of 1 mM Tris, 15% sucrose, pH 7.4 results in stable cakes upon storage at 2-8° C.

TABLE 4
Summary of the three months stability data at 2-8° C. and 25° C. as storage conditions

| | | Visual inspection | | | | | | | | | |
|---|---|---|---|---|---|---|---|---|---|---|---|
| | | Visual aspect | | | | Particle | | | | | |
| | | of cake and | | | | properties | | mRNA | | | |
| | | (reconstituted) | Reconstitution | | Visible | Z-ave | | Content | Encapsulation | | Osmolality |
| | | solution | time | | particles? | (nm) | PDI | (μg/mL) | efficiency (%) | pH | (mOsm/kg) |
| Liquid | T0 | NA | NA | | No | 99.91 | 0.07 | 102.22 | 96.37 | 7.31 | 891 |
| control | T1m | | NA | | No | 127.0 | 0.18 | 98.82 | 95.70 | 7.35 | 893 |
| at −80° C. | T2m | | NA | | No | 129.8 | 0.19 | 102.85 | 96.37 | 7.40 | 895 |
| (n = 1) | T3m | | NA | | No | 130.5 | 0.20 | 100.09 | 95.65 | 7.49 | 887 |
| FD cake | T0 | A | <30 | sec | No | 81.61 | 0.12 | 87.64 | 89.56 | 7.26 | 822 |
| at 2-8° C. | T1m | | <50 | sec | No | 80.67 | 0.12 | 76.96 | 89.94 | 7.29 | 819 |
| (n = 2) | T2m | | <30 | sec | No | 83.56 | 0.13 | 87.43 | 91.39 | 7.38 | 806 |
| | T3m | | 30 | sec | No | 80.85 | 0.12 | 98.85 | 92.11 | 7.45 | 800 |
| FD cake | T0 | A | <30 | sec | No | 81.61 | 0.12 | 87.64 | 89.56 | 7.26 | 822 |
| at 25° C. | T1m | | <50 | sec | No | 88.48 | 0.11 | 79.20 | 91.31 | 7.29 | 819 |

(n = 2)

A: Homogenous, coherent cake, no collapse or melting.

TABLE 5
Summary of the three months stability data at 2-8°
C. and 25° C. as storage conditions

| | | | Transfection HEK cells | | | | |
|---|---|---|---|---|---|---|---|
| | | Water | eGFP+ | eGFP+ | | | Integrity |
| | | content | cells | cells | MFI | MFI | testing |
| | | (%) | 0.5 μg (%) | 2 μg (%) | 0.5 μg | 2 μg | Degradation |
| Liquid | T0 | NA | 59.65 | 52.92 | 1187040 | 2380400 | No |
| control | T1m | NA | NA | NA | NA | NA | NA |
| at -80° C. | T2m | NA | NA | NA | NA | NA | NA |
| (n = 1) | T3m | NA | 40.28 | 52.96 | 292708 | 532226 | No |
| FD cake | T0 | 2.26 | 64.68 | 63.48 | 1444848 | 2773940 | No |
| at 2-8° C. | T1m | NA | NA | NA | NA | NA | NA |
| (n = 2) | T2m | 1.86 | NA | NA | NA | NA | NA |
| | T3m | 1.47 | 44.11 | 64.40 | 366864 | 824039 | No |
| FD cake | T0 | 2.26 | 64.68 | 63.48 | 1444848 | 2773940 | No |
| at 25° C. | T1m | NA | NA | NA | NA | NA | NA |
| (n = 2) | | | | | | | |

### Example 5—In Vivo Stability Testing

To investigate whether the storage conditions of a 3 mM Tris, 15% w/v sucrose composition after freeze-drying stored at 2-8° C., 25° C. or 30° C. for 0 months (T0m), 3 months (T3m) or 5 months (T5m) influenced the activity of the mRNA, an immunization study was performed wherein BALB/C mice were injected with LNP containing Sars-Cov2 Omicron spike mRNA or Fluc mRN. Mice received in total 2 intramuscular injections in the biceps of the hind limb with 50 μl of LNPs encapsulating Sars-Cov2 Omicron spike mRNA (5 μg) in TBS or buffer (negative control group) on day 1 and day 21 of the experiment. To monitor the development of immune response in these mice, a submandibular blood collection was performed on D20 and D35. The concentration of anti-Omicron IgG in serum was determined by ELISA.

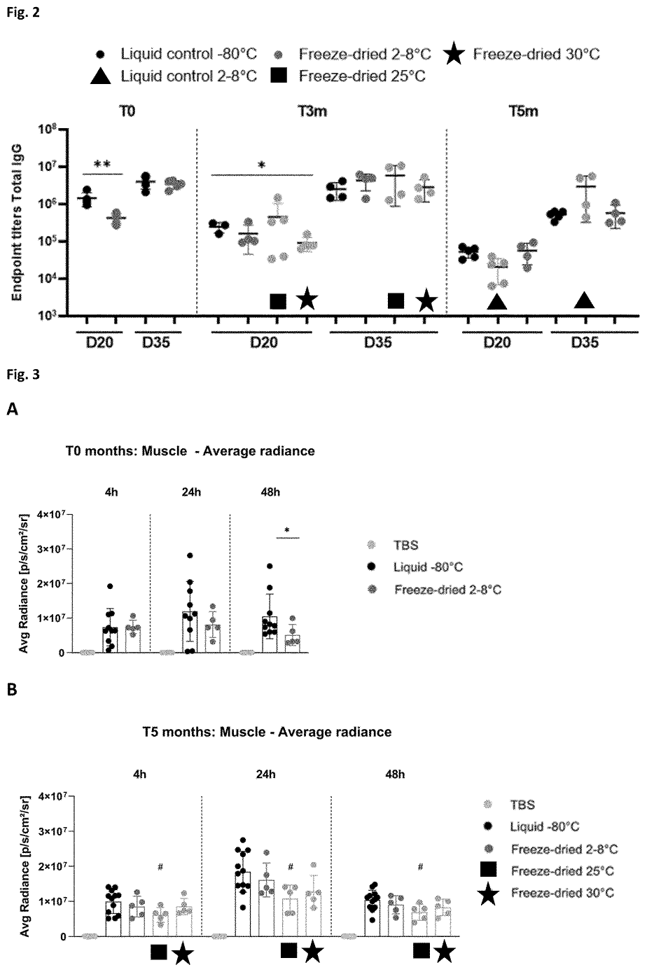

Overall, lyophilized samples demonstrated comparable IgG titers to the liquid control at −80° C. or the liquid form of lyophilized material (FIG. 2). At D20, some variability is detected, with lower expression in freeze-dried samples 2-8° C. at T0 and lower antibody titers for the freeze-dried 30° C. samples at T3m. Over time, it was observed that antibody titers slightly decrease with 10⁶-10⁷ total IgG at T0, 10⁵⁻⁶-10⁷ total IgG at T3m and 10⁵-10⁶ total IgG at T5m.

Next, expression levels via bioluminescence (BLI) were detected. Mice received one intramuscular injection (50 μl; 2 μg) with Fluc mRNA LNPs or TBS buffer (negative control group) in the biceps of the hind limb. At different time-points after injection (4 h, 24 h, 48 h), mice were placed in a bioluminescence scanner after intraperitoneal injection with luciferin and luciferase expression was measured. Highest expression was seen after 24 h for muscle especially for liquid −80° C. samples both at T0 (FIG. 3A) and T5m (FIG. 3B) as well as for lyophilized samples stored at temperature 2-8° C. It can be concluded that lyophilized samples, especially those of 2-8° C. and liquid −80° C. control samples showed comparable expression even after 5 months of storage.

### REFERENCES

Muramatsu H, Lam K, Bajusz C, Laczkó D, Karikó K, Schreiner P, Martin A, Lutwyche P, Heyes J, Pardi N, Lyophilization provides long-term stability for a lipid nanoparticle-formulated nucleoside-modified mRNA vaccine, Molecular Therapy (2022).
Zhao P., Hou X., Yan J. Long-term storage of lipid-like nanoparticles for mRNA delivery. Bioact Mater. 2020; 5(2):358-363.

1. A composition comprising: one or more RNA molecule(s), one or more lipid nanoparticle(s), sucrose, and Tris(hydroxymethyl)methylamine (TRIS);
wherein said composition is lyophilized; and wherein prior to lyophilization the concentration of TRIS in the composition is 3 mM or less and the concentration of sucrose is less than 20% (w/v).

2. The composition according to claim 1, wherein the concentration of sucrose in the composition prior to lyophilization is at least about 5% (w/v).

3. The composition according to claim 1, wherein the concentration of sucrose prior to lyophilization is from about 5% to about 20% (w/v).

4. The composition according to claim 1, wherein the concentration of TRIS in the composition prior to lyophilization is from about 0.01 mM to about 3 mM.

5. The composition according to claim 1, wherein said lipid nanoparticle(s) comprise a PEG lipid.

6. The composition according to claim 1, wherein said lipid nanoparticle(s) comprise from about 40 mol % to about 60 mol % of ionizable lipid, from about 5 mol % to about 15 mol % of phospholipid, from about 20 mol % to about 40 mol % of sterol, and at least 0.5 mol % of a PEG lipid.

7. The composition according to claim 1, wherein the composition prior to lyophilization further comprises water.

8. The composition according to claim 1, wherein said one or more RNA molecule(s) are linear or circular RNA molecule(s).

9. The composition according to claim 1, wherein said one or more RNA molecule(s) are mRNA molecule(s).

10. (canceled)

11. The composition according to claim 1, which is reconstituted before administration to a subject in need thereof.

12. The composition according to claim 11, wherein said reconstitution is performed using water or an aqueous solution of a salt.

13. The composition according to claim 11, which has a pH of about 6 to about 8.

14. A method of lyophilization of a composition comprising RNA molecules and lipid nanoparticles, said method comprising:
a. mixing RNA molecule(s), one or more lipid nanoparticle(s), a cryoprotectant and TRIS at a concentration of 3 mM or less in water; and
b. lyophilizing said composition comprising a freezing step, a primary drying step and a secondary drying step;
wherein said primary drying step is performed at a temperature below a glass transition temperature of a maximally freeze-concentrated solution (Tg′).

15. The method according to claim 14, wherein said primary drying step is performed at a temperature of from about −30° C. to about −50° C.

16. The composition according to claim 5, wherein the lipid nanoparticle(s) comprise DMG-PEG2000.

17. The composition according to claim 7, wherein the water is water for injection (WFI).

18. The composition according to claim 12, wherein the aqueous solution with a salt is TBS with 20 mM TRIS.
